(19) United States
(12) Patent Application Publication
(10) Pub. No.: US 2010/0047233 A1
(43) Pub. Date: Feb. 25, 2010

(54) TREATMENT OF POST-MENOPAUSAL AND POST-HYSTERECTOMY MEDIATED COGNITIVE DISORDERS

(76) Inventors: Mark A. Smith, Chagrin Falls, OH (US); Gemma Casadesus, Chagrin Falls, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/520,032

(22) PCT Filed: Dec. 18, 2007
(86) PCT No.: PCT/US07/87909

§ 371 Date: Jun. 18, 2009

Related U.S. Application Data
(60) Provisional application No. 60/875,441, filed on Dec. 18, 2006.

(51) Int. Cl. A61K 39/395; A61K 38/08; A61K 31/7105; A61P 25/28

(52) U.S. Cl. 424/130.1; 514/15; 514/44 A

(57) ABSTRACT

A method of treating or preventing post-menopausal or post-hysterectomy related cognitive decline in a subject includes administering to the subject a therapeutically effective amount of at least one physiologically acceptable non-estrogenic agent that reduces or eliminates gonadotropin and/or gonadotropin receptor levels in the subject.

### RELATED APPLICATION

This application claims priority from U.S. Provisional Application No. 60/875,441, filed Dec. 18, 2006, the subject matter, which is incorporated herein by reference.

### GOVERNMENT FUNDING

This invention was made with government support under Grant No. NIH-AG026151 awarded by The National Institutes of Health. The United States government has certain rights in the invention.

### FIELD OF THE INVENTION

The present invention relates to method of treating post-menopausal and post-hysterectomy mediated cognitive disorders.

### BACKGROUND

Normal aging is most often associated with cognitive impairments that result in decrements in the individual's quality of life and reduce his/her capability to live independently. Aging, in addition to its association to benign age-related cognitive decline, is also directly associated with an increased incidence of neurodegenerative diseases, such as Alzheimer disease (AD), the most common cause of dementia. Not surprisingly, the increasing life expectancy of our population has inevitably brought a higher incidence of cognitive symptomotology and age-related illnesses. In this regard, there are currently 4-5 million individuals in the United States affected with AD, however, this will rise to an estimated 14 million by 2050 unless successful treatments are developed. Unfortunately, thus far, little progress has been made with regards to deciphering the molecular mechanisms involved in cognitive decline nor in producing successful diagnostic tools or therapeutic strategies for age-related neurodegenerative diseases.

In normal aging humans and, to a more severe degree, individuals with AD episodic memories as well as working and spatial memories show progressive decline. Unfortunately, the mechanisms responsible for the types of behavioral declines and associated neuronal changes are yet to be elucidated. However, it is known that the hippocampus, a highly plastic area of the brain, is crucial in the modulation of cognition, specifically, episodic, spatial, and working memories. Notably, the hippocampus is one of the most age-sensitive areas in the brain and it is thought that the aging process greatly diminishes the plastic capabilities of this region and these declines lead to the age-related impairments in cognitive output.

While there are likely a number of age-related contributing factors involved in cognitive decline, one group of powerful modulators of cognition that also modulate hippocampal plasticity, are sex steroids such as estrogen and testosterone. In this regard, the role of sex steroids, and particularly estrogen, in age-related cognitive decline and AD have risen to prominence based on evidence showing, among other things, that estrogen deficiency, following menopause, may contribute to both benign cognitive decline as well as the etiology of AD, especially in women. These findings are further supported by epidemiological and observational studies indicating that hormone replacement therapy (HRT) lessens the risk of AD in post-menopausal women. However, recent contradictory reports from the Women's Health Initiative (WHI) study showed that HRT initiated in elderly post-menopausal women (ages 65 and above) does not improve cognitive performance and may actually increase the risk of developing AD. These data have caused reconsideration regarding the role of estrogen in age-related cognitive decline and the development of AD.

### SUMMARY OF THE INVENTION

The present invention relates to a method of treating or preventing post-menopausal or post-hysterectomy related cognitive decline in a subject. In the method, a therapeutically effective amount of at least one physiologically acceptable non-estrogenic agent that modulates or changes gonadotropin levels and/or gonadotropin receptor levels in the subject is administered to the subject.

In an aspect of the invention, the agent can be administered to the subject at an amount effective to reduce or eliminate gonadotropin levels, gonadotropin receptor levels, and/or amyloid-β levels in the brain.

In another aspect, the agent can be administered to subject to reduce or eliminate leutinizing hormone levels in the subject. The agent can also be administered during perimenopause to prevent cognitive decline.

In a further aspect, the agent can comprise at least one GnRH analog, GnRH antagonists, anti-GnRH antibody, GnRH receptor antagonist, anti-GnRH receptor antibody, LH antagonist, anti-LH antibody, LH receptor antagonist, anti-LH receptor antibody, human chorionic gonadotropin antagonist, anti-human chorionic gonadotropin antibody, human chorionic gonadotropin receptor antagonist, anti-human chorionic gonadotropin receptor antibody. An example of an agent can comprise leuprolide or a physically acceptable analogs and salts thereof. Another example of an agent can comprise interference RNA directed to mRNA that encodes brain derived gonadotropins and/or brain derived gonadotropin receptors.

The present invention further relates to a method of treating or preventing post-menopausal or post-hysterectomy related cognitive decline in a subject. In the method, a therapeutically effective amount of at least one physiologically acceptable non-estrogenic agent that reduces or eliminates brain derived gonadotropins and/or brain derived gonadotropin receptors in the subject is administered to the subject. The brain derived gonadotropin and/or gonadotropin receptor can comprise at least one of brain derived luteinizing hormone, brain derived luteinizing hormone receptor, brain derived human chorionic gonadotropin, and brain derived human chorionic gonadotropin receptor.

### BRIEF DESCRIPTION OF THE DRAWINGS

FIG. 1 is a graph illustrating cognitive performance as measured by a Y-maze. Percent number of alternations in a 5 minute trial for Tg-LHβ mice (Tg_LHβ) and Wild-type littermates (WT) (*=P<0.05).

FIG. 2 is a graph illustrating cognitive performance as measured by the Y-maze. Percent number of alternations in a 5 minute trial for LHRKO homozygous (−/−), heterozygous (+/−) and wild-type littermates (+/+) (*=P<0.05).

FIG. 3 is a plot illustrating Leuprolide, a gonadotropin-lowering drug, decreases brain Aβ levels in mice. C57Bl/6J mice (3 months old) were administered either vehicle or a slow release leuprolide acetate (1.5 mg/kg; intraperitoneal monthly) mixture at 0 and 4 weeks. Mice were euthanized at 0, 4, and 8 weeks, the brains were dissected, the frontal cortex tissues were homogenized and centrifuged, and the supernatant analyzed for Aβ 1-40 and Aβ 1-42 levels via an Aβ ELISA assay. Results are expressed as picograms/mg of total protein (mean±S.D., n=6 mice at each time point). *, p<0.05; **, p<0.0001 for differences between vehicle and treated animals at the same time point.

FIG. 4 illustrates that LH induces Aβ secretion and insolubility in neuroblastoma cells. Human M17 neuroblastoma cells were cultured and treated with 0, 10, and 30 mIU/ml of LH for 5 days. Media with corresponding LH concentrations were replaced every 2 days. The medium from each experiment was used to measure secreted Aβ 1-40 (A). Cell pellets were solubilized in Triton X-100 and centrifuged to generate soluble (B) and insoluble fractions (C). Aβ concentration is expressed as picograms/mg of total protein (mean±S.D.). Experiments were performed three times in duplicate (i.e. n=6, p<0.01). LH receptor expression pattern in human M17 neuroblastoma cells was determined by immunoblot analysis with (D) a rabbit polyclonal antibody against residues 15-38 and (E) a mouse monoclonal antibody (3B5). Arrows indicate the immature (59 kDa) full-length LH receptor.

FIG. 5 is a graph illustrating Y-Maze performance in Tg2576 mice after leuprolide acetate (n=8) or saline treatment (n=5) at baseline and after 3 months. Figure illustrates the mean % alternations expressed as % change from baseline.

(*) indicates significance at p<0.05.

FIG. 6 illustrates Aβ burden measured as % area stained in the entire hippocampus of 11 sections/brain/animal is significantly lower in animals treated with leuprolide acetate (n=8) compared to saline-treated animals (n=5, p<0.05). Representative image of Aβ burden in Tg2576 mice after saline (S) or leuprolide acetate (L). Scale bar, 200 μm.

FIG. 7 illustrates Leuprolide acetate treatment significantly reduces serum LH in Tg2576 mice (P<0.02). Inset: LHβ mRNA expression in mouse pituitary gland. Saline versus 6 and 8 week leuprolide acetate treatment.

FIG. 8 is a plot illustrating time course of serum LH following leuprolide acetate treatment.

FIG. 9 are graphs illustrating Y-maze performance after 3 months post-OVX or SHAM surgeries, Estrogen or placebo replaced (beginning at time of surgery) and treated with leuprolide acetate or saline. * Indicates a significant difference between saline and leuprolide acetate in the OVX+placebo group # indicates a significant difference between SHAM+saline and OVX+placebo+saline.

FIG. 10 are plots illustrating the length of time taken to find the invisible platform across three training days for OVX+estrogen (E) and placebo (P) replaced animals treated with leuprolide acetate (L) or saline (S) and sham operated animals (SHAM). (Significance indicated by: *P+S vs E+S; #=P+S vs P+L; $=P+S vs SHAM; %=P+S vs E+L; at p<0.05).

FIG. 11 are plots illustrating the distance swam in NE quadrant (A); Latency to enter NE quadrant (B); Number of platform crossings (C); Latency to enter platform location (D), during the probe trial for OVX+estrogen (E) and placebo (P) replaced animals treated with leuprolide acetate (L) or saline (S) and sham operated animals (SHAM).

FIG. 12 are graphs illustrating % Time spent in NE quadrant during the probe trial for OVX+estrogen (E) and placebo (P) replaced animals treated with leuprolide acetate (L) or saline (S) and sham operated animals (SHAM).

FIG. 13 are graphs illustrating Latency to enter NE quadrant and % Time spent in NE quadrant during the probe trial for animals treated with cetrorelix or saline and sham operated animals (SHAM).

FIG. 14 are plots illustrating the distance swam in NE quadrant during the trial for animals treated with cetrorelix or saline and sham operated animals.

### DETAILED DESCRIPTION

The present invention relates to a method of treating or preventing post-menopausal or post-hysterectomy related cognitive decline in a subject. The present invention is based on the discovery that increased gonadotropin levels (e.g., luteinizing hormone levels) in the brain of the subject in the presence of functional receptors may at least part be responsible for cognitive decline after menopause or a hysterectomy. The examples of the present invention suggest that modulation of gonadotropin (e.g., luteinizing hormone) or their receptors levels can be used as a therapeutic strategy for cognitive decline in age related neurodegenerative disease.

According to this invention, modulating (e.g., reducing) the level of gonatropins, such as luteinizing hormone and human chorionic gonadotropin (HCG), or their receptors in the brain of the subject prevents, treats, and/or inhibits cognitive decline in a post menopausal and post hysterectomy subject. Thus, the present invention entails a method of treating cognitive decline related to menopause and/or hysterectomy in a person suffering therefrom and a method of preventing cognitive decline in a person susceptible thereto by administration to the person a cognitive decline treatment-effective amount or a cognitive decline prevention-effective amount, respectively, of a non-estrogenic agent, which will modulate (e.g., change, alter, reduce) the level of gonadotropins or gonadotropin receptors in the person's brain. Among such agents are those selected from the group consisting of GnRH analogs and physiologically acceptable salts thereof, GnRH antagonists, GnRH receptor antagonists, gonadotropin antagonists (e.g., LH antagonists, human chorionic gonadotropin (HCG) receptor antagonists), gonadotropin receptor antagonists (e.g., LH antagonists or HCG antagonists), vaccines that stimulate production of anti-GnRH antibodies, anti-GnRH receptor antibodies, anti-gonadotropin antibodies, or anti-gonadotropin receptor antibodies, or conjunctive administrations of such compounds.

As indicated above, the invention entails treating post-menopausal and/or post-hysterectomy related cognitive decline in person. A person “suffering from post-menopausal and/or post-hysterectomy cognitive decline” is a person who has been diagnosed as having gradual cognitive decline, by a practitioner of at least ordinary skill in the art of clinically diagnosing, using methods and routines, such as those described below, that are standard in the art of such clinical diagnoses.

By “treating cognitive”, it is meant slowing or preventing the progression or worsening of the cognition that is now known to occur when untreated.

By “non-estrogenic” agents, it is meant agents that are not, respectively, estrogens or estrogen-like.

In accordance with the invention, cognitive decline in a post-menopausal and/or post-hysterectomy subject can be treated by administration to the subject any composition that reduces the subject's brain level of gonadotropin and/or gonadotropin receptor in an amount and for a duration effective to bring about such a reduction.

Further, in accordance with the invention, cognitive decline in a post-menopausal and/or post-hysterectomy subject can be prevented, or onset of clinical or behavioral manifestations delayed, in the subject by administration to the subject any composition that reduces the level of a gonadotropin or gonadotropin receptor in the brain of the subject in an amount and for a duration effective to bring about such a reduction to a level below which development of cognitive decline will not occur.

Reference herein to “level of a gonadotropin and/or gonadotropin receptor” in a person means concentration of the biologically active gonadotropin and/or gonadotropin receptor in the subject's brain. Typically, the level of a gonadotropin and/or gonadotropin receptor will be reduced by reducing the concentration of the gonadotropin and/or gonadotropin receptor itself. However, reducing the activity of the gonadotropin and/or gonadotropin, such as by binding it with an antibody that blocks the hormone's activity, even if the concentration of the gonadotropin and/or gonadotropin receptor in the brain remains the same, is considered reducing the level of the gonadotropin and/or gonadotropin receptor for purposes of the present application. The brain concentrations of gonadotropin and/or gonadotropin receptors in a human can be determined by any of a number of methods well known to the skilled.

As understood in the art, vaccines that stimulate production of antibodies can be employed to bind to gonadotropins (e.g., LH and HCG), gonadotropin receptors, GnRH and/or GnRH receptor block or at least substantially reduce their biological activities. Thus, vaccine-stimulated antibodies to gonadotropins (e.g., LH and HCG), gonadotropin receptors, GnRH and/or GnRH receptor can be employed in accordance with the invention to directly reduce the level of these proteins and thereby treat or prevent cognitive decline in post menopausal and post-hysterectomy subjects. Such antibodies to GnRH and/or GnRH receptor, by blocking its activity, will result in reduced levels of gonadotropins. These antibodies can be employed in accordance with the invention to reduce levels of gonadotropins and thereby to prevent or treat cognitive decline. Examples of such vaccines include the Talwar vaccine and a vaccine marketed under the tradename GONADIMMUNE by Aphton Corporation.

Antibodies for use in accordance with the invention may be made by conventional methods for preparation of vaccine antibodies for therapeutic use in humans. The vaccine-stimulated antibodies may be polyclonal and from any antibody-producing species, such as mice, rats, horses, dogs or humans. The antibodies may also be monoclonal from cultures of antibody-producing cells from an antibody-producing species, such as mice, rats, horses, dogs, and humans. The term “antibody” as used herein, unless otherwise limited, also encompasses antigen-binding fragments, such as Fab fragments, of intact antibodies. If an antibody is monoclonal but from cultured cells of a species other than human, the antibody may be “humanized” by conventional methods to make it more tolerable immunologically to a person treated therewith. Antibodies for use in accordance with the invention can also be made by conventional techniques using cultured cells, preferably human cells, that have been genetically engineered to make a desired intact antibody or antigen-binding antibody fragment.

Antibodies will be administered in accordance with the invention by any method known in the art for administering same but preferably by intravenous injection of a sterile aqueous solution of the antibody, together with standard buffers, preservatives, excipients and the like.

In an aspect of the invention, GnRH analogs and pharmaceutically acceptable salts thereof (e.g., leuprolide or goserelin, and especially leuprolide acetate and goserelin acetate) can be employed to reduce levels of gonadotropins to levels that are undetectable in the brain. Examples of GnRH analogs or salts thereof that may be employed in accordance with the invention include, among others, GnRH itself and its monoacetate and diacetate salt hydrates (Merck Index entry no. 5500) and the many analogs thereof that are known in the art. These include, for example, leuprolide and its monoacetate salt (Merck Index entry no. 5484, U.S. Pat. No. 4,005,063); the analogs of leuprolide with the D-leucyl residue replaced with D-α-aminobutyryl, D-isoleucyl, D-valyl or D-alanyl and the monoacetate salts thereof (U.S. Pat. No. 4,005,063); buserelin and its monoacetate salt (Merck Index entry no. 1527, U.S. Pat. No. 4,024,248); nafarelin and its monoacetate and acetate hydrate salts (Merck Index entry no. 6437, U.S. Pat. No. 4,234,571); deslorelin (Merck Index entry no. 2968); histrelin and its acetate salt (Merck Index entry no. 4760, U.S. Pat. No. 4,244,946); and goserelin and its acetate salt (Merck Index entry no. 4547, U.S. Pat. No. 4,100,274). For other GnRH analogs and salts thereof that can be used in accordance with the invention, see also U.S. Pat. No. 4,075,192, U.S. Pat. No. 4,762,717, and the U.S. patents cited at column 3, lines 49-54, of U.S. Pat. No. 4,762,717.

All of the U.S. patents cited herein, including those not cited specifically but cited at column 3, lines 49-54, of U.S. Pat. No. 4,762,717, and all of the Merck Index entries cited herein are incorporated herein by reference.

Administration of GnRH analogs in accordance with the invention will be by any method known in the art for administering same. Thus, administration may be by injection subcutaneously, intramuscularly or intravenously of a sterile aqueous solution which includes the analog together with buffers (e.g., sodium acetate, phosphate), preservatives (e.g., benzy alcohol), salts (e.g., sodium chloride) and possibly various excipients or carriers. In this connection, see, for example, Physician's Desk Reference, 51.sup.st Ed., Medical Economics Co., Montvale, N.J., U.S.A. (1997), pp. 2736-2746 (leuprolide acetate) and pp. 2976-2980 (goserelin acetate), which are also incorporated herein by reference.

The dose and dosage regimen for a particular composition used to carry out the invention with a particular patient will vary depending on the active ingredient and its concentration and other components in the composition, the route of administration, the gender, age, weight, and general medical condition of the patient, and whether the patient is already suffering from cognitive decline. The skilled medical practitioner will be able to appropriately prescribe dosage regimens to carry out the invention. It is preferred in carrying out the invention that the concentrations of gonadotropins and/or gonadotropin receptors be reduced to and maintained at levels that are as low as possible. It is usually preferred that the concentrations of gonadotropins be reduced to undetectable levels.

In one embodiment of carrying out the invention, a composition comprising a GnRH analog can be administered intramuscularly or subcutaneously as a depot composition from which release of the analog into the patient's system will be sustained over a long period, from about a week to about six months or more. This will maintain the concentration of gonadotropin in the brain of the subject at the low or undetectable level(s) as described above without the pain, cost and inconvenience of much more frequent (e.g., daily) administration. Such depot compositions of GnRH analogs are known and their preparation is well within the skill of the ordinarily person skilled in the art. See, e.g., Physician's Desk Reference, 51.sup.st Ed. pp. 2736-2746 and 2976-2980, cited above.

Information from data already available or easily obtained by routine experimentation on GnRH analogs in suppressing gonadotropin activity, those of ordinary skill can easily determine the dose and dosage regimens for any GnRH analog.

Also useful in carrying out the invention are agents that antagonize the activity of GnRH. Agents that block the receptors for GnRH or to directly inhibit production gonadotropins or both, will result in reduced levels of brain-derived and can be employed in accordance with the invention to treat or prevent cognitive decline in the brain. Examples of GnRH antagonists include, for example, citrorelix and abberelix as well as GnRH antagonist disclosed in U.S. Patent Publication No. 2007/0191403, which is herein incorporated by reference in it entirety.

Other agents that can be used in the methods of the present invention include gonadotropin antagonists, gonadotropin receptor antagonists, GnRH antagonists, and GnRH receptor antagonists as well as any agent or substance, which decreases the activity of gonadotropins and/or gonadotropin receptors in the brain. The gonadotropin antagonists, gonadotropin receptor antagonists, GnRH antagonists, and GnRH receptor antagonists may physically bind to the gonadotropin (e.g., LH or HCG) in the brain or to other components of the HCG pathway, such as GnRH, GnRH receptor, and gonadotropin receptors (e.g., LH receptor and HCG receptor), that facilitate cognitive decline in the brain.

Examples of LH antagonists include milrinone, cilostamide, amrinone, enoximone, CI-930, anagrelide, pimobendan, siguazodan (SKF-94836), lixazinone (RS-82856), imazodan (CI-914), indolidan (LY195115), quazinone, SKF 94120, Org 30029, adibendan (BM 14,478), APP 201-533, carbazeran, cilostazole, E-1020, IPS-1251, nanterinone (UK-61260), pelrinone, RMI 82249, UD-CG 212, bemarinone (ORF-16,600) CK-2130, motapizone, OPC-3911, Ro 13-6438, sulmazole, vesnarinone (OPC-8212), buquineran, DPN 205-734, ICI-170777, isomazole (LY175326), MCI-154, MS-857, OPC-8490, piroximone (MLD 19205), RS-1893, saterinone, ZSY-39, and ICI 118233 as well as compounds disclosed in U.S. Pat. No. 6,297,243, which is herein incorporated by reference in its entirety.

In another aspect of the invention, the gonadotropin antagonists, gonadotropin receptor antagonists, GnRH antagonists, and GnRH receptor antagonists comprise RNA interference (RNAi) reagents to induce knockdown of gonadotropins, gonadotropin receptors, GnRH antagonists, and GnRH receptors or of a protein which transduces gonadotropin antagonists, gonadotropin receptor antagonists, GnRH antagonists, and GnRH receptor antagonists. RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of in some instances as few as 21 to 22 base pairs in length. Furthermore, RNAi may be effected by introduction or expression of relatively short homologous dsRNAs. Indeed, the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs. For example, the siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR, which in its active form phosphorylates the translation initiation factor eIF2 to shut down all protein synthesis, and 2′,5′ oligoadenylate synthetase (2′,5′-AS), which synthesizes a molecule that activates Rnase L, a nonspecific enzyme that targets all mRNAs. The nonspecific pathway may represents a host response to stress or viral infection, and, in general, the effects of the nonspecific pathway are preferably minimized under preferred methods of the present invention. Significantly, longer dsRNAs appear to be required to induce the nonspecific pathway and, accordingly, dsRNAs shorter than about 30 bases pairs are preferred to effect gene repression by RNAi (see Hunter et al. (1975) J Biol Chem 250: 409-17; Manche et al. (1992) Mol Cell Biol 12: 5239-48; Minks et al. (1979) J Biol Chem 254: 10180-3; and Elbashir et al. (2001) Nature 411: 494-8). In mammalian cells, siRNAs are effective at concentrations that are several orders of magnitude below the concentrations typically used in antisense experiments (Elbashir et al. (2001) Nature 411: 494-8).

The double stranded oligonucleotides used to effect RNAi can be less than 30 base pairs in length and, for example, comprise about 25, 24, 23, 22, 21, 20, 19, 18 or 17 base pairs of ribonucleic acid. Optionally the dsRNA oligonucleotides of the invention may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells. Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan. Examples of dsRNAs may be synthesized chemically or produced in vitro or in vivo using appropriate expression vectors. Exemplary synthetic RNAs include 21 nucleotide RNAs chemically synthesized using methods known in the art (e.g. Expedite RNA phophoramidites and thymidine phosphoramidite (Proligo, Germany). Synthetic oligonucleotides are preferably deprotected and gel-purified using methods known in the art (see e.g. Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence.

If, for example, LH mRNA is the target of the double stranded RNA, any of the above RNA species will be designed to include a portion of a nucleic acid sequence that hybridizes, under stringent and/or physiological conditions to the LH mRNA. Likewise, if the target is the LH receptor mRNA, then any of the above RNA species will be designed to include a portion of a nucleic acid sequence that hybridizes, under stringent and/or physiological conditions to the corresponding mRNA sequence.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures that exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three-dimensional structure. A number of researchers have measured the binding energies of a large number of RNA duplex structures and have derived a set of rules which can be used to predict the secondary structure of RNA (see e.g. Jaeger et al. (1989) Proc. Natl. Acad. Sci. USA 86:7706 (1989); and Turner et al. (1988) Annu. Rev. Biophys. Chem. 17:167). The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerhead ribozyme compositions of the invention.

The dsRNA oligonucleotides may be introduced into the cell by transfection with an heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Further compositions, methods and applications of RNAi technology are provided in U.S. Pat. Nos. 6,278,039, 5,723,750 and 5,244,805, which are incorporated herein by reference.

Ribozyme molecules can be designed to catalytically cleave encoding mRNAs, or mRNAs encoding other members of the HCG pathway involved in gonadotropin activity and signalling (e.g., gonadotropin receptors, GnRH, and GnRH receptor). Ribozymes are enzymatic RNA molecules capable of catalyzing the specific cleavage of RNA. (For a review, see Rossi (1994) Current Biology 4: 469-471). The mechanism of ribozyme action involves sequence specific hybridization of the ribozyme molecule to complementary target RNA, followed by an endonucleolytic cleavage event.

While ribozymes that cleave mRNA at site specific recognition sequences can be used to destroy target mRNAs, the use of hammerhead ribozymes is preferred. Hammerhead ribozymes cleave mRNAs at locations dictated by flanking regions that form complementary base pairs with the target mRNA. The construction and production of hammerhead ribozymes is well known in the art and is described more fully in Haseloff and Gerlach (1988) Nature 334:585-591; and see PCT Appln. No. WO89/05852, the contents of which are incorporated herein by reference). Hammerhead ribozyme sequences can be embedded in a stable RNA such as a transfer RNA (tRNA) to increase cleavage efficiency in vivo (Perriman et al. (1995) Proc. Natl. Acad. Sci. USA, 92: 6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase III-mediated expression of tRNA fusion ribozymes are well known in the art (see Kawasaki et al. (1998) Nature 393: 284-9; Kuwabara et al. (1998) Nature Biotechnol. 16: 961-5; and Kuwabara et al. (1998) Mol. Cell 2: 617-27; Koseki et al. (1999) J Virol 73: 1868-77; Kuwabara et al. (1999) Proc Natl Acad Sci USA 96: 1886-91; Tanabe et al. (2000) Nature 406: 473-4). There are typically a number of potential hammerhead ribozyme cleavage sites within a given target cDNA sequence. Preferably the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA. In addition, ribozymes possess highly specific endoribonuclease activity, which autocatalytically cleaves the target sense mRNA. The present invention extends to ribozymes which hybridize to a sense mRNA encoding a gonadotropin, gonadotropin receptor, GnRH, and GnRH receptor.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme.

In certain embodiments, expression of the “target gene, whether it is gonadotropin, gonadotropin receptor, GnRH, and GnRH receptor gene or HCG pathway member, may be inhibited by an inhibitor RNA that is a single-stranded RNA molecule containing an inverted repeat region that causes the RNA to self-hybridize, forming a hairpin structure (a so-called “hairpin RNA” or “shRNA”). shRNA molecules of this type may be encoded in RNA or DNA vectors. The term “encoded” is used to indicate that the vector, when acted upon by an appropriate enzyme, such as an RNA polymerase, will give rise to the desired shRNA molecules (although additional processing enzymes may also be involved in producing the encoded shRNA molecules). The expression of shRNAs may be constitutive or regulated in a desired manner.

A double-stranded structure of an shRNA is formed by a single self-complementary RNA strand. RNA duplex formation may be initiated either inside or outside the cell. Inhibition is sequence-specific in that nucleotide sequences corresponding to the duplex region of the RNA are targeted for genetic inhibition. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Because 100% sequence identity between the RNA and the target gene is not required to practice the present invention, the invention has the advantage of being able to tolerate sequence variations that might be expected due to genetic mutation, strain polymorphism, or evolutionary divergence. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size. An endogenous RNA polymerase of the cell may mediate transcription of an shRNA encoded in a nucleic acid construct. The shRNA construct may also be synthesized by a bacteriophage RNA polymerase (e.g., T3, T7, SP6) that is expressed in the cell.

The foregoing description, discussion and scope of the invention are directed to those of ordinary skill in the treatment of actual or incipient AD. Accordingly, it is to be expected that the teachings herein will enable selection of specific agents and regimens for treatment within the scope of the appended claims.

### EXAMPLES

### Example 1

### Increases in Luteinizing Hormone are Associated with Declines in Cognitive Performance

In this study, we herein evaluated cognitive performance in two transgenic mouse strains, both with high LH but only one with functional LH receptors. LH receptors, as mentioned before, are found in high levels in the hippocampus, a region critical in the pathogenesis of AD. Therefore, testing LH-over-expressor mouse model such as the Tg-LHβ in addition to the LHRKO mouse models on a hippocampally-dependent task may allow us to determine whether cognitive changes are modulated as well as whether the changes are receptor specific in this region.

Additionally, an added bonus of these models is that Tg-LHβ and LHRKO have differential estrogen status. That is, while Tg-LHβ mice show high LH levels and high estrogen levels, LHKO mice show high LH levels but none-functional receptors and therefore below average levels of estrogen. This is important and relevant to AD since, as mentioned above, estrogen (declines) has been associated with AD/age-related cognitive declines.

### Methods

### Development and Characterization of Transgenic Lines

### Tg-LHβ Mice:

Transgenic mice expressing a chimeric LHβ subunit (LHβ) containing the C-terminal peptide of the human chorionic gonadotropin β subunit under the control of the αGSU promoter were previously described (Mann et al., 1999; Risma et al., 1995; Risma et al., 1997). All mice originated from one founder line and were F1 hybrids of CF-1 and FVB strains. Targeted expression of the LHβ chimera leads to elevated LH levels and infertility in female transgenic animals as well as increased estradiol and testosterone levels when compared to non-transgenic littermates (Mann et al., 2003).

### LH/hCG Receptor Gene Knockout Mice

LH/hCG receptors were disrupted by gene targeting in embryonic stem cells. The disruption resulted in infertility in both sexes and gonads and nongonadal tissues contained no receptor mRNA or receptor protein. The generation of this mouse is described in detail elsewhere (Lei et al., 2001). Briefly, a single gene with multiple transcription initiation sites present in the 5′-flanking region that encodes multiple transcripts and usually a single LH receptor protein was completely inactivated in the body using a targeting vector that deleted a part of the 5′-flanking region containing the promoter region and multiple transcription initiation sites, as well as most of exon 1. Disruption of the LH receptor gene led to increased levels of LH, decreased levels of estradiol and progesterone, and non-detectable levels of testosterone (Lei et al., 2001).

### Animals and Housing

For cognitive assessment (see below) we analyzed 9 transgenic LH-overexpressing female mice (Tg-LHβ) with an average age of 10 months and 15 age-matched non-transgenic littermates (average age 10 months). Additionally, we used 12 homozygous, 13 heterozygous transgenic LH receptor knock-out mice (LHRKO) and 8 age-matched wild-type littermates (average age 8 months). All animals were group housed, provided ad libitum access to food and water, and maintained on a 12 hr light/dark cycle. The Institutional Animal Care and Use Committee of Case Western Reserve University approved all animal studies.

### Behavioral Assessment—Y-Maze

To measure spontaneous alternation behavior and exploratory activity, a hippocampal-associated task, we used a Y-maze [32 cm (long)×10 cm (wide) with 26-cm walls]. Tg-LHβ and LHRKO animals were tested as previously described (Joseph et al., 2003). Briefly, each animal (randomized and investigator-blinded) was placed in one of three arms of the Y-maze (alternating arms across animals in each group) and each arm entry was recorded for duration of 5 minutes. An alternation was defined as 3 entries in 3 different arms (i.e., 1, 2, 3 or 2, 3, 1 etc). % number of alternations was calculated as (total alternations/total number of entries−2)×100. The maze was cleaned with ethanol between each animal to minimize odor cues.

### Statistical Analysis

A Student's T-test comparing the Y-maze performance in the Tg-LHβ mice versus aged-matched controls was used to determine statistical significance with assistance of statistical analysis software Sigmastat (SPSS, Inc., Chicago, Ill.). Statistical significance was determined at the p<0.05 level. A one-way analysis of variance (ANOVA) was used to determine Y-maze performance differences between homozygous (−/−), heterozygous (+/−) and wild-type (+/+) LHRKO mice. Multiple comparisons using the Fisher LSD test were carried out to determine statistically significant differences across each individual group.

### Results

Tg-LHβ mice demonstrated significant declines in Y-maze performance when compared to non-transgenic littermates (t_{1,21}=−6.712, p<0.05) in the absence of differences in overall exploratory activity (t_{1,21}=−1.626, p=0.119). In mice that harbored a disrupted LH receptor (LHRKO), there were no significant differences between homozygous and wild-type mice (t_{1,24}=0.316, p=1.0), however a statistically significant group effect was present (F_{2,31}=4.846, p<0.05) illustrating that heterozygous mice performed significantly worse than homozygous mice (t_{1,24}=2.923, p<0.05). As with Tg-LHβ, there were no differences in overall exploratory activity in the LHRKO mice across groups (F_{2,31}=0.895, p=0.419). To exclude possible sex or age-driven confounds, preliminary analyses prior to grouping animals across different gender and ages revealed no significant differences in Y-maze performance therefore data were collapsed across these variables for all subsequent analyses.

### Discussion

In this study we demonstrate that Tg-LHβ animals show declines in hippocampally-associated cognitive performance as measured by the Y-maze task. Previous reports reveal that LH is capable of modulating cognitive behavior and a more recent study demonstrates that experimental ablation of LH by a selective GnRH agonist (leuprolide acetate) improves Y-maze performance and decreases amyloid-β load in the hippocampus of APP transgenic mice. Given that LH is capable of crossing the blood-brain-barrier and that the highest level of LH receptors in the brain are localized to the hippocampus, an area that is highly vulnerable to both aging and AD, our data suggest that declines in hippocampally-related function may be associated with chronic LH elevation as seen in these mice or during menopause and AD in humans. With this in mind, the fact that Tg-LHβ animals sustain such high elevations in LH in addition to other elevated hormones (prolactin, corticosteroids, progesterone and testosterone) raises the possibility that the behavioral declines observed in these animals were mediated by indirect mechanisms rather than specifically via the LH receptor. To begin to address this issue, we also measured Y-maze performance in mice lacking functional LH receptors and found that homozygous knockout mice were indistinguishable from wild-type mice despite the fact that homozygous knockout mice show high elevations in LH; importantly, in the presence of reduced estrogen levels. On the other hand, our data also indicated that heterozygous knockout mice performed significantly worse than the two other groups in this task. While these results are somewhat surprising given that heterozygous mice are indistinguishable from wild-types in terms of hormonal levels (LH and estradiol), one possibility, which we are currently investigating, is that lower numbers of receptor under equal amounts of ligand leads to a potentiation response with receptors firing twice as much as they would normally do, thereby mimicking the findings observed for the Tg-LHβ animals.

Noteworthy, neither Tg-LHβ nor LHRKO animals showed declines in spontaneous alternation behavior in the absence of differences in overall exploratory activity. This supports the notion that the declines in Y-maze performance are hippocampal-specific rather than associated with a more general phenomenon such overall poorer health or tumor development in these animals. Furthermore, changes in estrogen levels in these animals were unlikely to be responsible for the cognitive changes observed in this study since Tg-LHβ mice show elevated, rather than diminished, levels of estrogen; LHRKO homozygous mice show decreased levels; and heterozygous knockout animals show equivalent levels of estrogen when compared to wild-type littermate controls. Therefore, and perhaps mimicking the situation in elderly-post-menopausal women undergoing HRT, estrogen levels appear not to be directly linked to declines in cognitive performance unless one takes into account the interrelationship with LH levels and LH receptor integrity. Notably, such an interrelation would explain the puzzling results described in the literature regarding the effectiveness of HRT to prevent cognitive decline and AD in post-menopausal women. Specifically, we suspect that increased dementia after HRT in elderly women (age 65 and above) may be attributable to the fact that while levels of estrogen were returned to pre-menopausal levels, levels of LH remain elevated and do not return to normal since the HPG axis feedback loop system, after years of chronic low estrogen and high gonadotropin levels, has already shut down. On the other hand, if HRT is started during peri- or early menopause, when the HPG axis feedback loop system is still functional, replacement of estrogen leads to the lowering of LH, and from epidemiological evidences, offers protection from age-related cognitive decline and AD.

In conclusion, our findings suggest that when examining declines in cognitive performance after menopause or during AD we should be careful to examine all the players involved in the equation rather than focusing on a single hormone. By solely focusing on estrogen we may be overlooking an ignored but very important partner, namely LH. In this regard, studies are currently underway to dissect the role of estrogen from that of LH using an ovariectomy as a model of menopause. More importantly, establishing the mechanism behind LH-related cognitive declines and targeting the release of LH may indeed be a successful strategy to prevent and forestall the progression of AD, illustrated by pre-clinical data using leuprolide acetate (Bowen et al., 2004; Casadesus et al., 2006) and a recently completed phase II clinical trial showing stabilization in cognitive impairment and activities of daily living in AD patients treated with high doses of leuprolide acetate. These promising findings support the importance of LH in AD and give way for an alternative and much needed therapeutic avenue for this insidious disease.

### Example 2

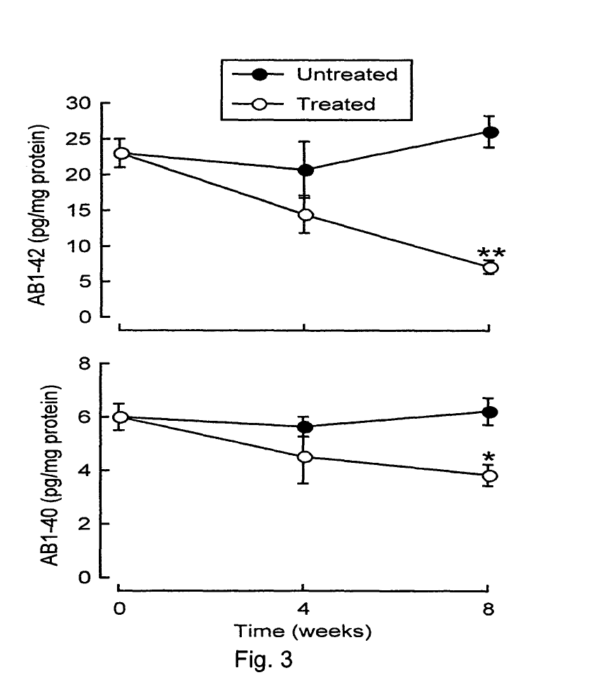

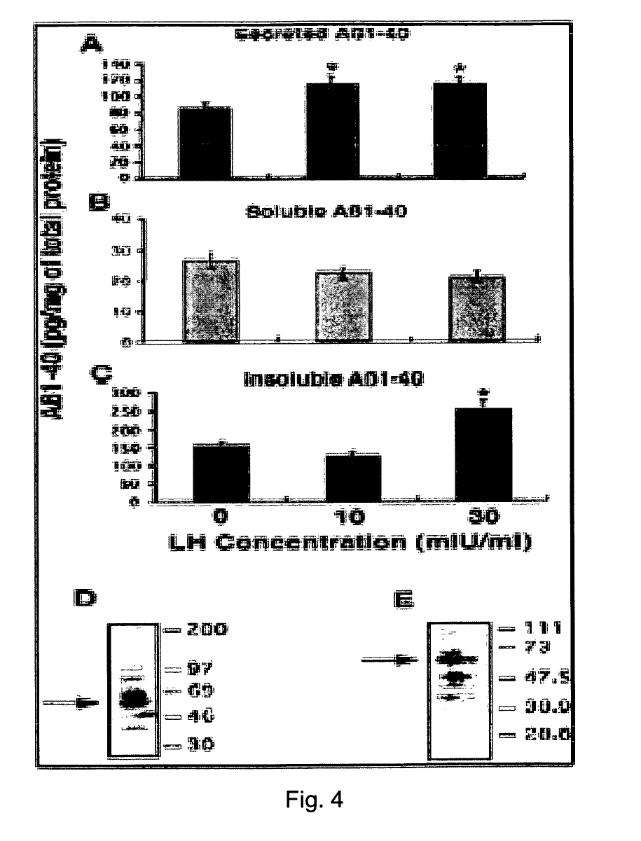

Based on these preliminary studies indicating that LH could be a factor in cognition and given that our group has previously reported that LH modulates amyloidogenic processing of AβPP (FIGS. 2 and 3), we evaluated the therapeutic potential of LH ablation, using a gonadotropin-releasing hormone analogue, leuprolide acetate, in aged (21 month old) Tg2576 female mice.

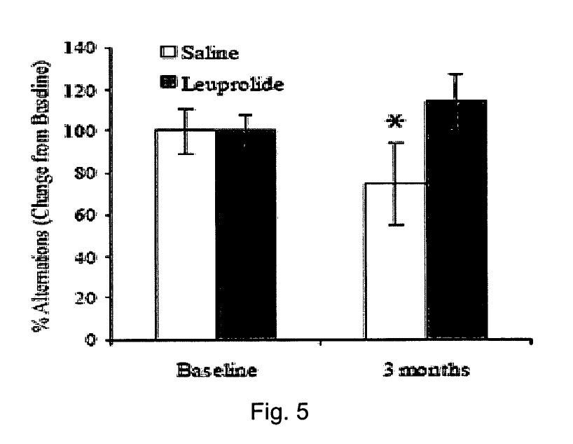

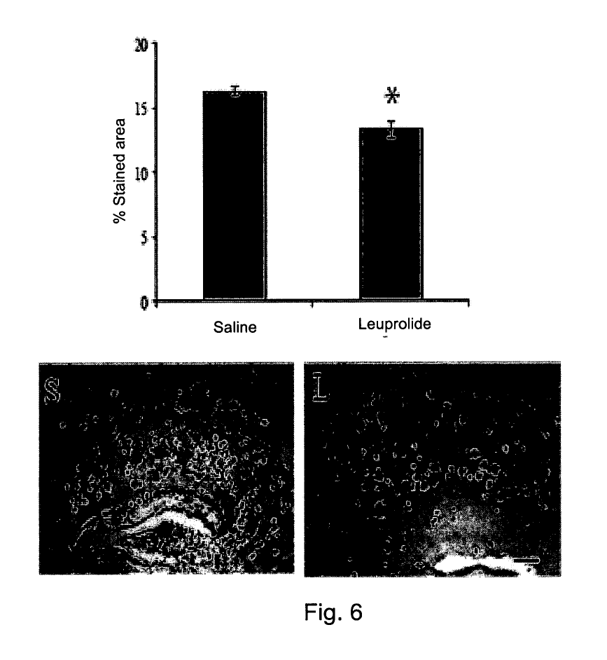

In this initial study we used aged animals to 1) circumvent the estrogen issue, since aged mice, like humans show estropause and 2) to examine the effects of LH ablation once the disease is well established. Our data indicates that LH ablation significantly attenuates cognitive decline (FIG. 5) (p<0.01) and decreases Aβ plaque load (p<0.05) (FIG. 6) as compared to placebo-treated animals. Therefore, our data suggests that, at least in aged AβPP transgenic mice, the positive effects of LH ablation override any negative effects of estrogen depletion. Importantly, while alternation behavior also depends on the innate tendency/preference of the animal to alternate, leading to the possibility that treatment, rather than improving/sustaining memory, could increase alternating preference, the fact that our data shows sustained rather than improved behavioral output in the treated animals compared to controls and the fact that treated animals did not show increases in overall arm entries nor any directional biases suggests that treatment did indeed sustain short-term memory rather than potentiate their preference to alternate.

Importantly, leuprolide acetate-mediated reductions of Aβ (p<0.05) was negatively correlated with improved cognition (r=−0.75, p<0.05). Such an assertion is in concert with data demonstrating that the modulation of estrogen in the AβPPP/PS-1 animal model of AD leads to improvements in cognitive behavior but, and unlike our findings, no changes in pathological features of AD. This discrepancy in results could be explained by a differential LH status in the animals of the two studies since while in our study we ablated both estrogen and LH concurrently, OVX leads to declines in estrogen but a rise in the levels of LH and administration of estrogen (c.f., HRT) does not decrease LH levels beyond baseline. Therefore, one possibility is that it is only the decrease in estrogen when it is coupled with an increase in LH that leads to behavioral impairments and it is only the ablation of LH that leads to changes in Aβ pathology in these mice.

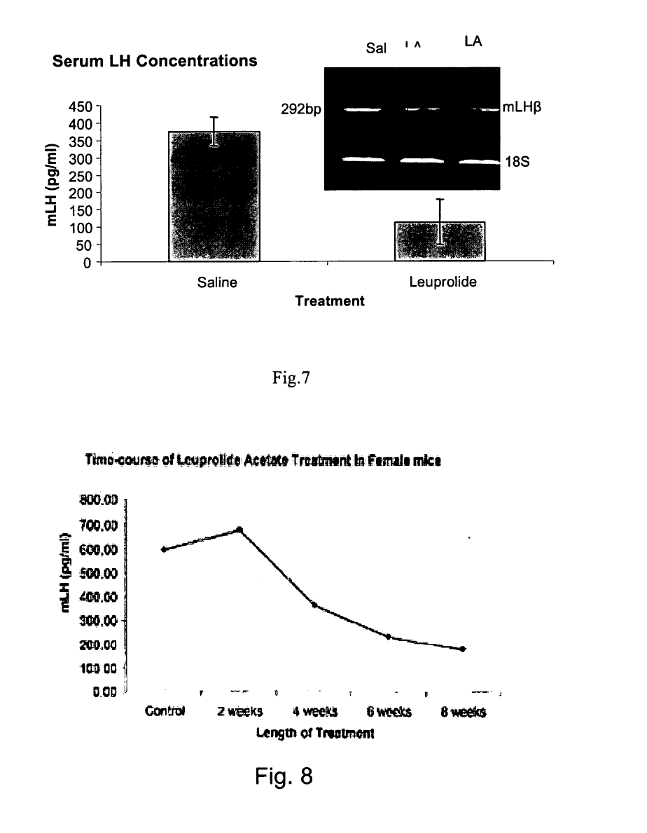

Treatment with leuprolide acetate causes a significant decline in serum LH (FIG. 7) paralleling a clear decrease in the expression of LHβ mRNA in the pituitary (FIG. 7 inset) in the mice used for this proposal. Similarly, a time-course (up to 8 weeks) following leuprolide acetate treatment depicts the classical LH modulation pattern by GnRH agonists (FIG. 8). These data support the notion that treatment with leuprolide acetate in mice follows the same pattern as treatment in human patients with regards to serum LH level modulation.

### Example 3

We present preliminary data gathered from a pilot study and that focuses on the effects of leuprolide acetate on cognition in ovariectomized animals with or without estrogen replacement immediately after surgery.

For this pilot 2 month old C57/B6 female mice from Jackson Labs, were ovariectomized (n=40) them and either replaced them with 90-day time-release estrogen (n=20) or placebo pellets (Innovative research of America, F1) (n=18). 24 hours after surgeries and pellet implantations half of the animals were treated with either 0.9% saline an the other half with leuprolide acetate (7.5 mg/kg) for 3 months in an identical fashion to that previously reported (Casadesus et al., 2006). In addition we added a SHAM ovariectomized group that received saline and a placebo pallet (n=10). During the last two weeks of the study all animals have been tested for Y-maze and Morris Water Maze performance.

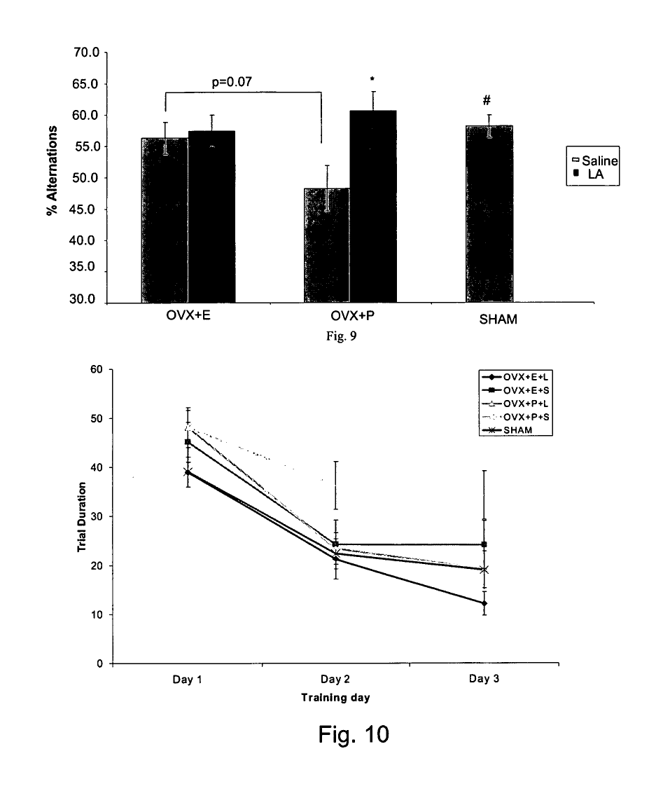

This supplemental material using Y-maze as a broad measure of cognitive function supports the tenants of our proposal as we have found that leuprolide treatment in OVX placebo-implanted mice was effective at improving OVX-dependent cognitive declines. Specifically, we found a group difference of drug treatment (leuprolide vs saline) when comparing OVXed animals that were replaced with estrogen or placebo (F=5.145; p=0.02). That is, leuprolide acetate significantly improved Y-maze performance as compared to saline-treated animals. Post-hoc analyses demonstrated that that this significant improvement of cognitive output was specific to the placebo replaced group (t=2.939; p=0.005). Additionally, our data indicates a strong trend (t=1.841; p=0.07) towards significance when comparing saline-treated estrogen-replaced animals versus saline-treated placebo replaced OVX animals (FIG. 9).

We feel that lack of statistical significance in the surgical parameter was likely due to the relatively small n number in each group rather than lack of success of the surgery protocol for two reasons: 1) that we observed a significant decline in cognitive performance in the OVX+Placebo+Saline group compared to the SHAM+sal group (F=5.802; p=0.02) and 2) that we observed a significant increase in body weight in the OVX+placebo group as compared to OVX+estrogen group (F=5.649; p<0.001) irrespective of drug treatment. Importantly, the OVX+estrogen replaced group was not different from the SHAM operated group, hence indicating that indeed estrogen replacement had an effect. We will be able to further confirm this point when we sacrifice the animals and send out blood samples for estrogen and gonadotropin measurements.

TABLE 1
Body weight table after 3 months post-surgery +
replacelement + drug treatment

| | | |
|---|---|---|
| OVX + Estrogen | OVX + Placebo | SHAM |

| LA | Saline | LA | Saline | Saline |
|---|---|---|---|---|
| 22.1 | 23.5 | 26.3 | 28.5 | 22.8 |
| 0.2 | 0.4 | 1.6 | 1.3 | 0.4 |

As mentioned before, all mice also underwent MWM testing, a measurement of hippocampal function based on the capacity of the animal to find a hidden platform under water by remembering and using spatial cues in the environment. Importantly, these data further support our findings in the Y-maze. To this end, here we demonstrate that OVX in our protocol was successful at producing cognitive decline in our mice (F=5.308; p=0.03) when compared SHAM operated animals and that this cognitive decline was rescued by estrogen replacement (F=4.073; p=0.05), as measured by the length of time taken to locate the invisible platform across days (FIG. 10). More importantly, our data also indicates that leuprolide acetate was as effective as estrogen in rescuing OVX-associated cognition decline (F=5.176; p=0.028) and that overall, independent of replacement regiment, animals treated with leuprolide acetate learned at a faster rate than did animals treated with saline (F=3.783; p=0.027). Importantly, as indicated below (FIG. 10) all groups showed a progressive decline in time spent to find the hidden platform (F=82.378; p<0.001), thus Confirming that indeed the training worked and the OVX+placebo treated with saline performed significantly more poorly compared to the rest of the groups on days 2 and 3.

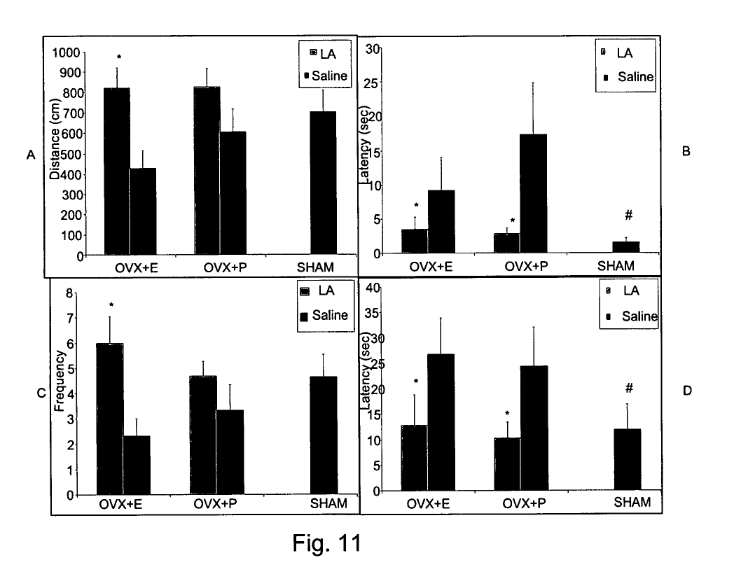

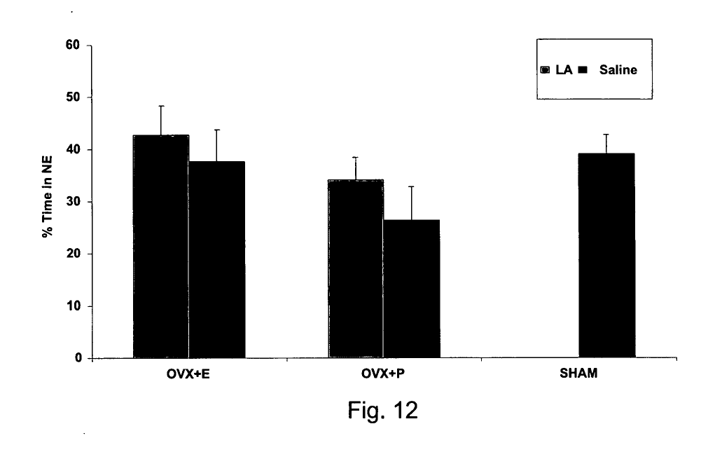

Additionally, to further determine the memory function and training success in the animals we measured the capacity of the animals to retain the information learned using a probe trial. In this regard, at the end of the last day of training we removed the platform and let the animals swim for 1 minute. We found that leuprolide treated animals swam longer distances in the quadrant that had previously held the platform (NE quadrant) (F=9.655; p=0.003) entered that quadrant earlier (F=4.427; 0.041), crossed the invisible platform region more earlier (5.028; p=0.005) and crossed it more times (F=9.115; p=0.004) regardless of replacement regiment (FIG. 11) and that, there was a strong trend of replacement regiment towards significance for % time spent in the NE trial (FIG. 12).

We feel that this data strongly further supports our hypothesis as evidenced by the fact that ablation of gonadotropins has a significant positive impact on cognition in ovariectomized/estropausal animals. This data supports our findings in previous published literature using an AD transgenic model.

### Example 4

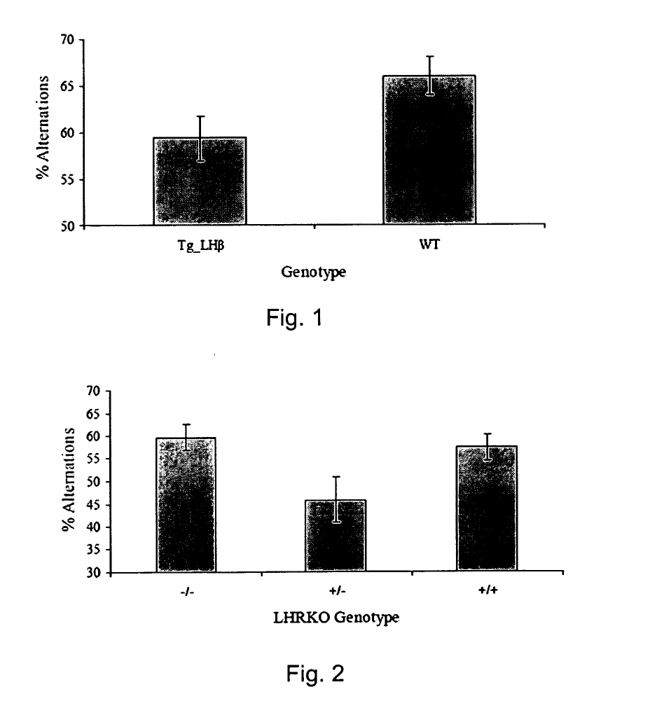

GnRH antagonists such as Cetrorelix pomoade suppress GnRH function without an initial stimulatory effect. GnRH antagonists are newer drugs, however, these compounds seem to be as effective as established therapy, but with shorter treatment times and less side-effects. Importantly, treatment with antagonists and agonists can, at least partially, dissect the role of LH because of the different pharmacokinetics of these compounds onto LH release (FIG. 1B). Specifically, how they bind to the receptor, competitively (leuprolide) vs non-competitive blocking (Cetrorelix) and the pattern of activation of Ca++ on the GnRH receptor (leuprolide activates the release of Ca++ but Cetrorelix does not). Because both cetrorelix and leuprolide seem effective at modulating cognition, it seems likely that the effects are produced via LH rather than GnRH since Cetrorelix literally shuts down the GnRH receptor.

We present preliminary data gathered from a pilot study and that focuses on the effects of cetrorelix on cognition in ovariectomized animals with or without estrogen replacement immediately after surgery. For this pilot 2 month old C57/B6 female mice were ovariectomized, as described above, and then treated with either 0.9% saline and the other half with cetrorelix. In addition we added a SHAM ovariectomized group that received saline and a placebo pallet (n=10). During the last two weeks of the study all animals have been tested for Morris Water Maze performance.

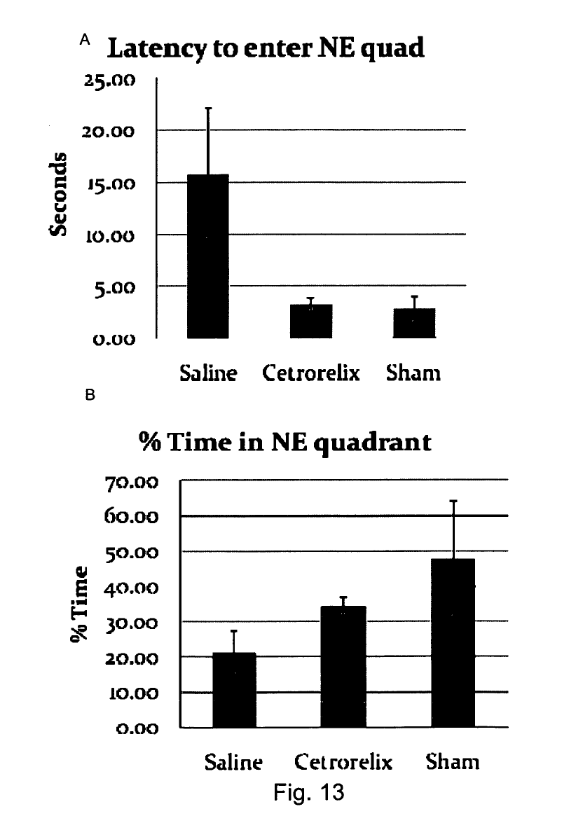

We have found that cetrorelix treatment in OVX placebo-implanted mice was effective at improving OVX-dependent cognitive declines (FIG. 13). Specifically, we found a group difference of drug treatment (cetrorelix vs saline) when comparing OVXed animals that were acetate was as effective in rescuing OVX-associated cognition decline and that overall, independent of replacement regiment, animals treated with cetrorelix learned at a faster rate than did animals treated with saline). Importantly, as indicated (FIG. 13) all groups showed a progressive decline in time spent to find the hidden platform.

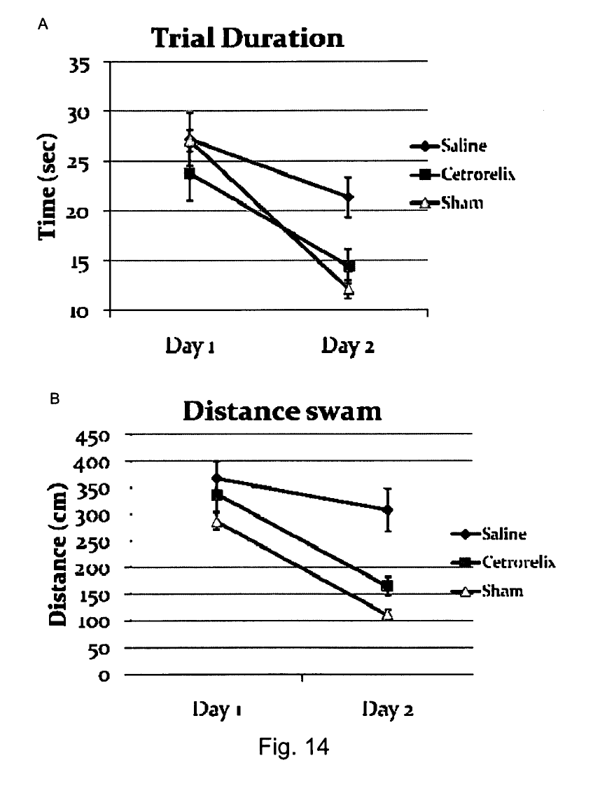

Additionally, to further determine the memory function and training success in the animals we measured the capacity of the animals to retain the information learned using a probe trial. In this regard, at the end of the last day of training we removed the platform and let the animals swim for 1 minute. We found that cetrorelix treated animals swam longer distances in the quadrant that had previously held the platform (NE quadrant) (FIG. 14).

From the above description of the invention, those skilled in the art will recognize, or be able to ascertain using no more than routine experimentation, numerous equivalents to the compounds and methods of use thereof described herein. Such equivalents are considered to be within the scope of this invention and are covered by the following claims. All patents, publications, and references cited in the present application are herein incorporated by reference in their entirety.

1-9. (canceled)

10. A method of treating post-menopausal or post-hysterectomy related cognitive decline in a subject, the method comprising:
administering to the subject a therapeutically effective amount of at least one physiologically acceptable non-estrogenic agent that reduces or eliminates gonadotropin and/or gonadotropin receptor levels in the subject.

11. The method of claim 10, the agent being administered to the subject at an amount effective to reduce or eliminate brain derived gonadotropin and/or gonadotropin levels.

12. The method of claim 10, the agent reducing or eliminating leutinizing hormone levels in the subject.

13. The method of claim 10, the agent being administered to the subject at an amount effective to reduce or eliminate amyloid-β levels in the brain.

14. The method of claim 10, the agent reducing the level of at least one of GnRH or GnRH receptor in the subject.

15. The method of claim 10, the agent being administered to the subject during perimenopause to prevent cognitive decline

16. The method of claim 10, the agent comprising at least one GnRH analogs, GnRH antagonists, anti-GnRH antibody, anti-GnRH receptor antibody or, gonadotropin antagonists, gonadotropin receptor antagonists, anti-gonadotropin antibody, or anti-gonadotropin receptor antibody.

17. The method of claim 16, the agent comprising leuprolide or a physically acceptable analogs and salts thereof.

18. The method of claim 16, the agent comprising interference RNA directed to mRNA that encodes gonadotropin, gonadotropin receptor in the brain.

19. A method of treating post-menopausal or post-hysterectomy related cognitive disorders in a subject, the method comprising:
administering to a subject a therapeutically effective amount of at least one physiologically acceptable non-estrogenic agent that reduces or eliminates leutinizing hormone levels in the subject, the agent comprising at least one of GnRH analogs, GnRH antagonists, anti-GnRH antibody, anti-GnRH receptor antibody or, gonadotropin antagonists, gonadotropin receptor antagonists, anti-gonadotropin antibody, or anti-gonadotropin receptor antibody.

20. The method of claim 19, the brain derived gonadotropin and/or gonadotropin receptor comprising at least one of brain derived luteinizing hormone, brain derived luteinizing hormone receptor, brain derived human chorionic gonadotropin, and brain derived human chorionic gonadotropin receptor.

21. The method of claim 19, the agent being administered to the subject at an amount effective to reduce or eliminate brain derived gonadotropin and/or gonadotropin levels.

22. The method of claim 19, the agent reducing or eliminating leutinizing hormone levels in the subject.

23. The method of claim 19, the agent being administered to the subject at an amount effective to reduce or eliminate amyloid-β levels in the brain.

24. The method of claim 19, the agent reducing the level of at least one of GnRH or GnRH receptor in the subject.

25. The method of claim 19, the agent being administered to the subject during perimenopause to prevent cognitive decline

26. The method of claim 19, the agent comprising leuprolide, cetrorelix, physically acceptable analogs and salts thereof.

27. The method of claim 19, the agent comprising interference RNA directed to mRNA that encodes gonadotropin, gonadotropin receptor in the brain.
